(19) United States
(12) Patent Application Publication
(10) Pub. No.: US 2005/0192251 A1
(43) Pub. Date: Sep. 1, 2005

(54) Water soluble bioactive fraction isolated from gum resin exudate of Boswellia serrata, process for isolation thereof composition containing said fraction and use thereof

(76) Inventors: Sunil Kumar Banerjee, Jammu (IN); Om Parkash Gupta, Jammu (IN); Subhash Chandra Taneja, Jammu (IN); Bishan Datt Gupta, Jammu (IN); Vijay Kumar Sethi, Jammu (IN); Avtar Singh Anand, Jammu (IN); Lila Ram Manhas, Jammu (IN); Rajinder Kumar Gupta, Jammu (IN); Sarang Bani, Jammu (IN); Surjeet Singh, Jammu (IN); Neelam Sharma, Jammu (IN); Sukhdev Swami Handa, Jammu (IN)

Correspondence Address: MORGAN & FINNEGAN, L.L.P., 3 World Financial Center, New York, NY, 10281-2101, US

(21) Appl. No.: 11/115,823

(22) Filed: Apr. 27, 2005

Related U.S. Application Data
(62) Division of application No. 10/113,519, filed on Mar. 28, 2002.

(51) Int. Cl. A61K 31/736; C08B 37/00

(52) U.S. Cl. 514/54; 536/114

(57) ABSTRACT

The present invention relates to a water-soluble bioactive fraction obtained from the gum resin exudate of Boswellia serrata and to a process for the preparation thereof. The present invention also relates to a process for the isolation of a water soluble bioactive fraction containing a mixture of potassium and calcium salts of polysaccharides composed of units of arabinose, galactose and D-glucuronic acid, having marked anti-inflammatory and anti-arthritic activities from the gum resin exudate of Boswellia serrata and to the use thereof in the treatment of arthritis in the form of a pharmaceutical composition containing the bioactive fraction.

### FIELD OF THE INVENTION

The present invention relates to a water-soluble bioactive fraction obtained from the gum resin exudate of Boswellia serrata. The present invention also relates to a process for the preparation of a water-soluble bioactive fraction from the gum resin exudate of Boswellia serrata (Salai guggal) belonging to the family Burseraceae. More particularly, the present invention relates to a process for the isolation of a water soluble bioactive fraction containing a mixture of potassium and calcium salts of polysaccharides composed of units of arabinose, galactose and D-glucuronic acid, having marked anti-inflammatory and anti-arthritic activities from the gum resin exudate of Boswellia serrata.

### BACKGROUND OF THE INVENTION

The gum resin exudate of Boswellia serrata (Salai guggal) has traditionally been used in the Ayurvedic system of medicine in India for treatment of inflammatory diseases [Hagers Handbuch der pharmazeut. Praxis, (1972), 4^{th} Ed., Vol. 111, pp. 491, Springer-Verlag, Berlin, Heidelberg, New York].

An alcoholic extract of the petroleum ether washed gum resin exudate of Boswellia serrata (AEPWR), available in the Indian market under the trade name “Sallaki” since 1982, was found to have anti-inflammatory activity (Singh, G. B., Singh, S. and Bani, S., Drugs of Today, 1996, 32, 109-112; Singh G. B. and Atal, C. K., Agents and Actions, 1986, 18, 407). This is also available in Switzerland as H-15. It has a novel mode of inhibitory action on the formation of the lipoxygenase product, leukotriene B (LTB₄) (Ammon, H. P. T., Mack, T., Singh, G. B. and Safayhi H., Planta Medica, 1991, 57, 203-207).

β-Boswellic acid (BA) and its derivatives, 11-keto-β-boswellic acid (KBA) and acetyl-11-keto-β-boswellic acid (AKBA) have been isolated as active constituents from alcoholic extract of petroleum ether washed gum resin (AEPWR) and show prominent anti-inflammatory and anti-arthritic properties (Sharma, M. L., Bai S. and Singh, G. B., Int. J Immunopharmacol., 1989, 11, 647-652; Singh, G. B., Singh, S. and Bani S., Drugs of the Future, 1993, 18, 307-309) with a selective inhibitory action on the formation of leukotriene B (LTB₄) (Safayhi H. et al., J. Pharmacol. Exp. Ther., 1992, 261, 1143-1146). The gum resin exudate of B. serrata (SG) on extraction with organic solvents like methanol or ethanol, or petroleum ether followed by methanol or ethanol yields about 60-65% of extract. The marc weighing about 35-40% of the gum resin (SG) is composed of water-soluble constituents, dust and plant residues.

A polysaccharide was isolated from the gum resin exudate of B. Serrata and characterized as a 4-0-methyl-glucuronoarabinogalactan (Sen, A. K., Das, A. K., Banerji, N and Vignon, M. R., Carbohydrate Research, 1992, 223, 321-327). This product, a pure polysaccharide, was isolated from the defatted oleogum resin in about 9% yield by extraction with water followed by dialysis and a number of chromatographic separations using DEAE-Sephacel and Sephadex G-100. This implies that the yield of this pure polysaccharide is much less than 9% on the weight of the gum resin.

While focus has been given to extraction of bioactive fractions from the gum resin exudates to obtain sallaki or H-15, no attention has been given to the waste fraction obtained in such processes. It is also believed that the polysaccharide fraction is less than 9% by weight of the total gum resin.

It is therefore important to attempt to obtain useful bioactive fractions from the waste obtained after the production of Sallaki or H-15.

### OBJECTS OF THE INVENTION

The main object of the present invention is to provide a process of preparation of a novel water soluble bioactive fraction from Boswellia serrata.

Another object of the present invention is to provide a novel water soluble bioactive fraction containing water soluble compounds having marked anti-inflammatory and anti-arthritic activities.

### SUMMARY OF THE INVENTION

Accordingly the present invention provides a novel bioactive fraction obtained from the gum resin exudate of Boswellia serrata comprising polysaccharides with at least 50 per cent neutral sugars (taken as galactose) consisting of galactose and arabinose and D-glucuronic acid.

In another embodiment of the invention, the fraction prepared comprises a mixture of salts of calcium 1.4 to 2.1% and potassium 0.12 to 0.20%.

The present invention also relates to a process for the preparation of a water soluble novel bioactive fraction from gum resin exudate of Boswellia serrata comprising extracting the gum resin exudate or defatted gum resin exudate with an alkanol to produce marc, extracting the marc with water and precipitating the polysaccharide fraction from the aqueous extract by addition of alcohol and purifying the bioactive fraction.

In one embodiment of the invention, the bioactive fraction obtained comprises polysaccharides with at least 50 per cent neutral sugars (taken as galactose) consisting of galactose and arabinose and D-glucuronic acid.

In another embodiment of the invention, the alkanol is selected from ethanol and methanol.

In another embodiment of the invention, the marc left after extraction with alcohol is extracted with water at room temperature.

In another embodiment of the invention, the aqueous extract is precipitated with alcohol to get the crude polysaccharide fraction and purified by repeating this step.

In another embodiment of the invention, the bioactive composition is collected by filtration and dried under vacuum at temperatures below 50° C.

In another embodiment of the invention, the fraction prepared comprises a mixture of salts of calcium 1.4 to 2.1% and potassium 0.12 to 0.20%.

The present invention also relates to a composition for the treatment of arthritis comprising a pharmaceutically effective amount of a bioactive fraction obtained from Boswellia serrata comprising polysaccharides with at least 50 per cent neutral sugars (taken as galactose) consisting of galactose and arabinose and D-glucuronic acid in a pharmaceutically acceptable carrier.

In another embodiment of the invention, the fraction prepared comprises a mixture of salts of calcium 1.4 to 2.1% and potassium 0.12 to 0.20%.

The present invention also relates to a method for the treatment of arthritis comprising administering a pharmaceutically effective amount of a bioactive fraction obtained from Boswellia serrata comprising polysaccharides with at least 50 per cent neutral sugars (taken as galactose) consisting of galactose and arabinose and D-glucuronic acid in a pharmaceutically acceptable carrier.

In another embodiment of the invention, the fraction prepared comprises a mixture of salts of calcium 1.4 to 2.1% and potassium 0.12 to 0.20%.

The present invention also relates to the use of a bioactive fraction obtained from Boswellia serrata comprising comprising polysaccharides with at least 50 per cent neutral sugars (taken as galactose) consisting of galactose and arabinose and D-glucuronic acid to prepare a pharmaceutical composition for the treatment of arthritis.

### DETAILED DESCRIPTION OF THE INVENTION

The bioactive product obtained in this invention is the total polysaccharide fraction and is obtained in about 15% yield on the weight of the oleoresin, the isolation of which has been achieved using a facile process. The fraction is obtained from a waste product viz., the material discarded after production of Sallaki or H-15. In addition the fraction also contains potassium and calcium salts of glucuronoarabinogalactose as ascertained by its hydrolysis and incineration. Hitherto all the pharmaceutical products based on the oleo-gum resin of B. serrata are insoluble in water, whereas the fraction of the invention is water soluble and its isolation was the result of an investigation based on activity guided separation of the constituents of the gum resin of B. serrata. The marc left after extraction of the gum resin with alcohol (ethanol or methanol) or petroleum ether followed by alcohol, the latter being used for the production of commercial “Sallaki” or H-15 of Switzerland, was processed for isolation of polysaccharide fraction with a view to evaluating it for anti-inflammatory activity. By extracting with water the marc left after extracting the gum resin exudate of B. serrata with alcohol (ethanol or methanol) followed by precipitation of the aqueous extract with ethanol it was possible to isolate the crude polysaccharide. The crude polysaccharide was purified by redissolving it in water and reprecipitating with ethanol. This process was repeated once again to give water soluble bioactive composition in 14-16 per cent yields containing polysaccharide with at least 50 per cent neutral sugars (taken as galactose/glucose) as determined by phenol-sulphuric acid method, acid hydrolysis of which yielded galactose, arabinose and D-glucuronic acid. This bioactive polysaccharide composition, has anti-inflammatory activity comparable with that of “boswellic acids” (total acids from SG) or “Sallaki” and anti-arthritic activity stronger than that of boswellic acids or “Sallaki” developed as anti-inflammatory/anti-arthritic agents from the gum resin exudate of Boswellia serrata.

The bioactive fraction (composition) prepared by the process of the present invention has the following characteristics:
i) it is a white or almost white (pale yellow) solid;
ii) it is completely soluble in water;
iii) on incineration it gave 4.5 to 6% ash which was found to be a mixture of carbonates of potassium and calcium (corresponding to ˜0.17% potassium and ˜1.8% calcium). The ash dissolved partly in water but completely in dilute hydrochloric acid;
iv) on hydrolysis with 2N trifluoroacetic acid it gave arabinose, galactose and D-glucuronic acid.
v) the fraction of the invention was found to contain 50 per cent neutral sugars as determined by phenol-H₂SO₄ method (Dubois, M. et al. Anal. Chem. 1956, 28, 350-356).

The detection of potassium and calcium in the ash obtained on incineration of the fraction of the invention indicated that either these metals are present in the fraction as impurities in the form of salts of inorganic acids or as salts of the D-glucuronic acid moiety. The former, i.e., the presence of potassium and calcium salts of inorganic acids in the fraction of the invention was ruled out as the ash obtained on incineration of the fraction in about 5 per cent yield consisted of potassium carbonate (˜0.3 per cent) and calcium carbonate (˜4.6 per cent) only and no other anions such as chloride, sulphate or nitrate could be detected in the ash. The composition of the ash obtained on incineration corresponds to ˜0.17% potassium and 1.8% calcium in the fraction of the invention. Thus the fraction is a mixture of calcium and potassium salts of the polysaccharides, composed of units of galactose, arabinose and D-glucuronic acid, in the ratio of ˜10 to ˜1.

On comparative evaluation of antiarthritic activities of boswellic acids, the fraction of the invention and 1:1 mixture of boswellic acids and the fraction of the invention in adjuvant induced developing and developed arthritis in rats based on inhibition (in injected paw) of edema in treated groups as compared to the control groups it was observed that in doses of 200 mg/kg p.o. boswellic acids gave an inhibition of 31.20% whereas the fraction of the invention and the mixture of boswellic acids and the fraction of the invention gave inhibitions of 35.47% and 36.22% respectively on the 13th day. On the 28th day at the same dose levels the percentage inhibitions with boswellic acids, the fraction of the invention and the mixture of boswellic acids and the fraction of the invention were 21.02, 25.15 and 23.72 respectively. Thus, the fraction of the invention and boswellic acids on the combination in equal doses showed additive and not synergistic effect.

A study on the effects of boswellic acids and the fraction of the invention on total leucocytes count and volume of pleural fluid after intrapleural carrageenan injection revealed that percentage inhibitions with boswellic acids and the fraction of the invention in pleural fluid were 6.52 and 7.56 respectively and in TLC 28.08 and 32.28 respectively. The fraction of the invention which like boswellic acids acts as a 5-lipoxygenase inhibitor is free from any ulcerogenic effects, as expected, which makes it a very good therapeutic agent for the treatment of arthritis.

The process of the present invention is illustrated by the following examples, which are, however, not to be construed to limit the scope of the present invention.

All the samples of the bioactive composition on analysis were found to conform to the characteristics described above.

### EXAMPLE 1

The finely ground gum resin exudate of Boswellia serrata (one year old sample, i.e. one year after collection-20 mesh powder, 1 kg) was charged in a percolator, treated with ethanol (3 litres) and left overnight. The percolate was drained and the marc was extracted twice at room temperature, each time with 3 litres of ethanol. The marc (370 g) (the drug left after extraction with ethanol) was air dried and extracted with water (1.85 litres) at room temperature for 8 hours. It was filtered through a cloth and the residue again treated with water (750 ml). After 8 hours it was filtered and combined filtrates on centrifuging gave a clear supernatant liquid (somewhat viscous) (2.25 litres). It was cooled and alcohol (4.5 litres) was added to it with stirring. The mixture was left in a fridge. The precipitate (almost white), which separated out was collected by filtration. It was again dissolved in water (1.5 litres) and the aqueous solution precipitated by adding alcohol (3 litres). It was filtered and one more purification by dissolving it in water (1.4 litres) and reprecipitating with alcohol (2.8 litres) afforded the bioactive composition. It was collected by filtration and dried in a vacuum desiccator at room temperature, yield 159 g (15.9%).

### EXAMPLE 2

The finely ground gum resin exudate of Boswellia serrata (one year old sample, 1 kg) was charged in a percolator and extracted thrice with petroleum ether (60-80° C.) at room temperature using 2 litres of the solvent each time. The marc was air-dried and extracted thrice with alcohol (3×2 litres). The residue (350 g after air-drying) left after extraction with alcohol was extracted with water (1.4 litres) for 8 hours at room temperature and filtered through a muslin cloth. The residue was washed with water (200 ml). The filtrate was combined with the washing and centrifuged. To the clear supernatant (1.1 litres) alcohol (2.2 litres) was added. Precipitate was dissolved in water and reprecipitated with alcohol. One more purification as described in Example 1 gave the bioactive composition, yield 142 g.

### EXAMPLE 3

Powdered gum resin exudate of Boswellia serrata (a three-year-old sample, 1 kg) was extracted in a percolator with petroleum ether (3×2 litres). The defatted gum thus obtained (715 g) was extracted thrice with alcohol in the percolator (3×2 litres). The marc was first extracted with water (1.4 litres) at room temperature and subsequently with 700 ml and then with 450 ml of water. The combined aqueous extracts (expressed through muslin cloth) (1.8 litres) were centrifuged to get a clear supernatant (1.7 litres) which on dilution with alcohol (3.4 litres) deposited pale brown solid. The mixture was left overnight in a fridge and the solid collected by filtration. It was dissolved in water (1.4 litres) and precipitated by adding alcohol (2.8 litres) to the aqueous solution. This process was repeated once more when the bioactive composition was obtained as a very light brown (almost white) solid, yield 149 g.

### EXAMPLE 4

Powdered gum resin exudate of Boswellia serrata (one year old resin, 0.5 kg) was thrice extracted with methanol (3×1.5 litres) at room temperature in a percolator. The marc 175 g was air-dried and extracted twice at room temperature with water using 700 ml of water for the first extraction and then 200 ml for the subsequent extraction. The combined aqueous extracts were clarified by centrifugation and the clear extract (˜600 ml) was precipitated by adding alcohol. The light brown solid which separated out on the leaving the mixture overnight was collected by filtration and purified by dissolving it in water (500 ml) and precipitating with alcohol (1 litre). The process was repeated once more when the bioactive composition separated out as a light yellow (off white) solid. It was collected by filtration and dried in vacuum; yield 73 g.

Advantages:

1. The bioactive fraction prepared in the present invention has immense potential in therapeutic use from a waste product viz., discarded material left after production of Sallaki or H-15.
2. Its anti-arthritic activity is more than that of well known anti-arthritic drug viz., Boswellic acids.
3. It does not have any ulcerogenic effects that are commonly associated with anti-inflammatory and anti-arthritic drugs.

1-11. (canceled)

12. Method for the treatment of arthritis comprising administering a pharmaceutically effective amount of a bioactive fraction obtained from Boswellia serrata comprising polysaccharides with at least 50 per cent neutral sugars (taken as galactose) consisting of galactose and arabinose and D-glucuronic acid in a pharmaceutically acceptable carrier.

13. Method as claimed in claim 12 wherein the fraction as prepared comprises a mixture of salts of calcium 1.4 to 2.1% and potassium 0.12 to 0.20%.

14. Method as claimed in claim 12 wherein the dose of said fraction administered comprises 15 to 25 mg/kg of body weight of the subject.

15-17. (canceled)
